SEQUENCE LISTING (60 sequences)

SEQ ID NO: 1 - protein, 14 aa, Artificial sequence
  Synthetic peptide
KAFAKLAARLYRKA

SEQ ID NO: 2 - protein, 14 aa, Artificial sequence
  Synthetic peptide
AAFAKLAARLYRKA

SEQ ID NO: 3 - protein, 14 aa, Artificial sequence
  Synthetic peptide
KAFAALAARLYRKA

SEQ ID NO: 4 - protein, 8 aa, Artificial sequence
  Synthetic peptide
YKKIIKKL

SEQ ID NO: 5 - protein, 14 aa, Artificial sequence
  Synthetic peptide
KHKGRDVILKKDVR

SEQ ID NO: 6 - protein, 18 aa, Artificial sequence
  Synthetic peptide
YEKPGSPPREVVPRPRPV

SEQ ID NO: 7 - protein, 16 aa, Artificial sequence
  Synthetic peptide
KNNNQKSEPLIGRKKT

SEQ ID NO: 8 - protein, 20 aa, Artificial sequence
  Synthetic peptide
RYVVLPRPVCFEKGMNYTVR

SEQ ID NO: 9 - protein, 8 aa, Artificial sequence
  Synthetic peptide
KPGPRGQR

SEQ ID NO: 10 - protein, 5 aa, Artificial sequence
  Synthetic peptide
KDPKR

SEQ ID NO: 11 - protein, 4 aa, Artificial sequence
  Synthetic peptide
RSRK

SEQ ID NO: 12 - protein, 11 aa, Artificial sequence
  Synthetic peptide
YGRKKRRQRRR

SEQ ID NO: 13 - protein, 11 aa, Artificial sequence
  Synthetic peptide
YARKARRQARR

SEQ ID NO: 14 - protein, 11 aa, Artificial sequence
  Synthetic peptide
YARAAARQARA

SEQ ID NO: 15 - protein, 11 aa, Artificial sequence
  Synthetic peptide
YARAARRAARR

SEQ ID NO: 16 - protein, 11 aa, Artificial sequence
  Synthetic peptide
YARAARRAARA

SEQ ID NO: 17 - protein, 11 aa, Artificial sequence
  Synthetic peptide
YARRRRRRRRR

SEQ ID NO: 18 - protein, 11 aa, Artificial sequence
  Synthetic peptide
YAAARRRRRRR

SEQ ID NO: 19 - protein, 14 aa, Artificial sequence
  Synthetic peptide
KAFAKLAARLYRAA

SEQ ID NO: 20 - protein, 14 aa, Artificial sequence
  Synthetic peptide
KAFAKLAAQLYRKA

SEQ ID NO: 21 - protein, 15 aa, Artificial sequence
  Synthetic peptide
WKAFAKLNCRLYRKA

SEQ ID NO: 22 - protein, 17 aa, Artificial sequence
  Synthetic peptide
AKLNCRLYRKANKSSKL

SEQ ID NO: 23 - protein, 21 aa, Artificial sequence
  Synthetic peptide
FHRRIKALHRRVKIFHRRIKA

SEQ ID NO: 24 - protein, 15 aa, Artificial sequence
  Synthetic peptide
WKAFAKLAARLYRKA

SEQ ID NO: 25 - protein, 21 aa, Artificial sequence
  Synthetic peptide
FHRRIKAFHRRIKAFHRRIKA

SEQ ID NO: 26 - protein, 7 aa, Artificial sequence
  Synthetic peptide
FHRRIKA

SEQ ID NO: 27 - protein, 13 aa, Artificial sequence
  Synthetic peptide
GRKKRRQRRRPPQ

SEQ ID NO: 28 - protein, 12 aa, Artificial sequence
  Synthetic peptide
AYARAAARQARA

SEQ ID NO: 29 - protein, 34 aa, Artificial sequence
  Synthetic peptide
DAATATRGRSAASRPTERPRAPARSASRPRRPVE

SEQ ID NO: 30 - protein, 27 aa, Artificial sequence
  Synthetic peptide
GWTLNSAGYLLGLINLKALAALAKKIL

SEQ ID NO: 31 - protein, 18 aa, Artificial sequence
  Synthetic peptide
KLALKLALKALKAALKLA

SEQ ID NO: 32 - protein, 21 aa, Artificial sequence
  Synthetic peptide
KETWWETWWTEWSQPKKKRKV

SEQ ID NO: 33 - protein, 16 aa, Artificial sequence
  Synthetic peptide
KAFAKLAARLYRKAGC

SEQ ID NO: 34 - protein, 16 aa, Artificial sequence
  Synthetic peptide
KAFAKLAARLYRAAGC

SEQ ID NO: 35 - protein, 16 aa, Artificial sequence
  Synthetic peptide
AAFAKLAARLYRKAGC

SEQ ID NO: 36 - protein, 16 aa, Artificial sequence
  Synthetic peptide
KAFAALAARLYRKAGC

SEQ ID NO: 37 - protein, 16 aa, Artificial sequence
  Synthetic peptide
KAFAKLAAQLYRKAGC

SEQ ID NO: 38 - protein, 16 aa, Artificial sequence
  Synthetic peptide
AGGGGYGRKKRRQRRR

SEQ ID NO: 39 - protein, 7 aa, Artificial sequence
  Synthetic peptide
FHRRIKA

SEQ ID NO: 40 - protein, 7 aa, Artificial sequence
  Synthetic peptide
LNRRAKL

SEQ ID NO: 41 - protein, 7 aa, Artificial sequence
  Synthetic peptide
LHRRVKI

SEQ ID NO: 42 - protein, 7 aa, Artificial sequence
  Synthetic peptide
FHRRIKL

SEQ ID NO: 43 - protein, 34 aa, Artificial sequence
  Synthetic peptide
DAATATRGRSAASRPTERPRAPARSASRPRRPVG

SEQ ID NO: 44 - protein, 16 aa, Artificial sequence
  Synthetic peptide
RQIKIWFQNRRMKWKK

SEQ ID NO: 45 - protein, 5 aa, Artificial sequence
  Synthetic peptide
KKKKX

SEQ ID NO: 46 - protein, 5 aa, Artificial sequence
  Synthetic peptide
RRRRX

SEQ ID NO: 47 - protein, 14 aa, Artificial sequence
  Synthetic peptide
FHRRIKAFHRRIKA

SEQ ID NO: 48 - protein, 28 aa, Artificial sequence
  Synthetic peptide
FHRRIKAFHRRIKAFHRRIKAFHRRIKA

SEQ ID NO: 49 - protein, 16 aa, Artificial sequence
  Synthetic peptide
KAFAKLAARLYRKAGC

SEQ ID NO: 50 - protein, 16 aa, Artificial sequence
  Synthetic peptide
AAFAKLAARLYRKAGC

SEQ ID NO: 51 - protein, 16 aa, Artificial sequence
  Synthetic peptide
AGGGGYGRKKRRQRRR

SEQ ID NO: 52 - protein, 17 aa, Artificial sequence
  Synthetic peptide
GKAFAKLAARLYRKAGC

SEQ ID NO: 53 - protein, 17 aa, Artificial sequence
  Synthetic peptide
AAAFAKLAARLYRKAGC

SEQ ID NO: 54 - protein, 17 aa, Artificial sequence
  Synthetic peptide
AKAFAALAARLYRKAGC

SEQ ID NO: 55 - protein, 12 aa, Artificial sequence
  Synthetic peptide
GYGRKKRRQRRR

SEQ ID NO: 56 - protein, 12 aa, Artificial sequence
  Synthetic peptide
AYGRKKRRQRRR

SEQ ID NO: 57 - protein, 17 aa, Artificial sequence
  Synthetic peptide
AKAFAKLAARLYRKAGC

SEQ ID NO: 58 - protein, 16 aa, Artificial sequence
  Synthetic peptide
KAFAKLAARLYRKAGC

SEQ ID NO: 59 - protein, 16 aa, Artificial sequence
  Synthetic peptide
AGGGGYGRKKRRQRRR

SEQ ID NO: 60 - protein, 4 aa, Artificial sequence
  Synthetic peptide
RGDS

(19) United States
(12) Patent Application Publication
(10) Pub. No.: US 2003/0190364 A1
(43) Pub. Date: Oct. 9, 2003

(54) Biological affinity based delivery systems

(76) Inventors: Alyssa Panitch, Higley, AZ (US); Brandon Seal, Mesa, AZ (US)

Correspondence Address: GALLAGHER & KENNEDY, P. A., 2575 E. CAMELBACK RD. #1100, PHOENIX, AZ, 85016, US

(21) Appl. No.: 10/405,339

(22) Filed: Apr. 1, 2003

Related U.S. Application Data
(60) Provisional application No. 60/369,568, filed on Apr. 1, 2002.

(51) Int. Cl. A61K 38/17; A61K 31/727; A61K 9/14; A61K 31/737; A61K 31/728

(52) U.S. Cl. 424/488; 514/54; 514/56; 514/12

(57) ABSTRACT

The present invention provides compositions for drug delivery, comprising a polymer network; a plurality of polysaccharide binding (PB) polypeptides, wherein the plurality of PB polypeptides are covalently bound to the polymer network, but wherein the PB polypeptides do not serve to covalently cross-link the polymer network; and negatively charged polysaccharides non-covalently bound to the plurality of PB polypeptides; as well as methods for making and using the compositions.

### CROSS REFERENCE

This application claims priority to U.S. Provisional Patent Application Serial No. 60/369,568 filed Apr. 1, 2002, incorporated by reference herein in its entirety.

### FIELD OF THE INVENTION

The present invention relates to the fields of polymer chemistry, polysaccharides, polypeptides, drug delivery devices, and methods for drug delivery.

### BACKGROUND OF THE INVENTION

Polymeric-based systems for in vivo delivery of therapeutic agents are currently the subject of active research. While such polymeric-based systems exist, they generally suffer from one or more drawbacks. Many covalent polymeric networks cannot be delivered in situ but require implantation. Covalent polymeric networks that can be formed in situ require chemical, light or enzymatic initiation, which requires time; although the time for polymerization can be relatively brief (i.e. several seconds to a few minutes), any time spent during polymerization allows the components of a delivery system to diffuse away. Furthermore, chemical and photo-initiators are often toxic, while enzymatic depend on enzyme kinetics. Additionally, covalent delivery vehicles cannot degrade without the incorporation of hydrolytic or enzymatic degradation sites. The degradation of networks incorporating chemistries for hydrolytic degradation is nonspecific and can be difficult to control. The degradation of networks incorporating chemistries for enzymatic recognition and cleavage depends on, among other things, enzyme diffusion into and through the network and local regulation of enzyme expression.

Fewer examples exist of physical polymeric systems. Often, physical systems require time (sometimes several hours) to form. Furthermore, the formation of physical systems can involve temperatures, pH and salt concentrations that are outside the range of physiological conditions. Similarly, delivery systems formed via covalent cross-linking of a polymeric material by polysaccharide binding polypeptides do not immediately form networks. Furthermore, covalent gels cannot be reformed if the covalent chemical bonds are broken, and they cannot change their shape within a dynamic, remodeling environment such as those that exist within normal, healing or regenerating tissues.

An ideal physical system based on biological affinity of the components would form a gel-like material immediately at physiologically relevant temperature, pH and salt concentrations. Such an ideal physical system would be capable of reforming after a mechanical or environmental insult or perturbation, and would also be capable of modifying their shape to accommodate alterations in in vivo geometry or surroundings. Since such physical systems would assemble in a manner that mimics assembly of the extracellular matrix (physical gelation), they are more appropriate for use in vivo use than covalently crosslinked gels. Therapeutics with affinity for a component of this physical system could be sequestered within the system and released based on the relative affinity between the therapeutic and the system, as well as diffusion.

Thus, such delivery systems based on biological affinity, and methods for drug delivery using such systems, are needed in the art.

### SUMMARY OF THE INVENTION

The present invention provides drug delivery compositions, and methods for making and using them, that do not suffer from the drawbacks of previous drug delivery devices.

The present invention provides compositions for drug delivery, comprising:

a) a polymer network;

b) a plurality of polysaccharide binding (PB) polypeptides, wherein the plurality of PB polypeptides are covalently bound to the polymer network, but wherein the PB polypeptides do not serve to covalently cross-link the polymer network; and

c) negatively charged polysaccharides non-covalently bound to the plurality of PB polypeptides.

In further embodiments, the compositions comprise a therapeutic of interest. Further aspects of the invention include methods for making the compositions, and methods for using the compositions for drug delivery.

### BRIEF DESCRIPTION OF THE FIGURES

FIG. 1. The results of the FITC-labeled TAT release experiment are shown over a course of 28 hours following the addition of the TAT to the gels. The letters in the legend correspond to sample groups in the following manner: A—HBD high-TAT; B—HBD low-TAT; C—HBD high-heparin-TAT; D—HBD low-heparin-TAT; E—dextran-heparin-TAT.

FIG. 2 is a graph depicting diffusion of heparin-based peptides out of heparin-based gels.

FIG. 3 is a graph depicting the diffusion of heparin-binding peptides from heparin-based gels.

FIG. 4 is a chart depicting the relative heparin affinity of heparin-binding peptides.

FIG. 5 is a graph of the dynamic mechanical properties of heparin-based compositions.

FIG. 6 is a graph of the dynamic mechanical properties of dextran sulfate-based compositions.

FIG. 7 is a graph of the repeated dynamic mechanical properties of a heparin-based composition.

FIG. 8 is a graph of a temperature sweep of a heparin-based composition.

FIG. 9 is a graph of a temperature sweep of a dextran sulfate-based composition.

FIG. 10 is a graph showing a comparison between transition temperature of heparin and dextran sulfate-based compositions.

### DETAILED DESCRIPTION OF THE INVENTION

The present invention provides drug delivery compositions, and methods for making and using them, that do not suffer from the drawbacks of previous drug delivery devices. The currently disclosed compositions form a gel-like material upon mixture at physiologically relevant temperature, pH and salt concentrations, and are capable of reforming after a mechanical or environmental insult or perturbation. The compositions of the invention are also capable of modifying their shape to accommodate alterations in in vivo geometry or surroundings. Thus, the compositions assemble in a manner that mimics assembly of the extracellular matrix (physical gelation), and as such are more appropriate for in vivo use than covalently crosslinked gels.

The compositions are adaptable and can be used for the release of virtually any therapeutic agent. The affinity of the therapeutic agents to the negatively charged polysaccharides can be tailored to meet the desired release rates. Furthermore, since these compositions assemble in a manner that mimics assembly of the extracellular matrix (physical gelation), they are more physiologically appropriate for in vivo use than covalently crosslinked gels.

In one aspect, the present invention provides novel compositions comprising

a) a polymer network;

b) a plurality of polysaccharide binding (PB) polypeptides, wherein the plurality of PB polypeptides are covalently bound to the polymer network, but wherein the PB polypeptides do not serve to covalently cross-link the polymer network; and

c) negatively charged polysaccharides non-covalently bound to the plurality of PB polypeptides.

Binding of the negatively charged polysaccharide to the PB polypeptides serves to coordinate the composition through one or more non-covalent interactions including but not limited to hydrogen bonding, hydrophobic interactions, ionic bonds, and physical entanglement, resulting in the formation of the compositions of the invention, which can be in the form of a viscous solution or a physical gel. Various physiological factors, such as proteases, heparinases (and/or other factors that serve to degrade the polymer, polypeptide or negatively charged polysaccharide used in the composition), and diffusion lead to loss of coordination of the composition over time. By varying the specific components of the composition, and/or their relative concentrations, the composition can be designed to retain coordination for longer or shorter periods of time, depending on a user's needs. As a result, when a therapeutic agent is added to the composition (see below), its release profile from the composition can be optimized based on the specific needs of the therapeutic and the patient.

The composition is a “physical gel” when the storage modulus (G′) is equal to or larger than the loss modulus (G″). The composition is a viscous solution when G′ is lower than G″. It should be noted that the composition can comprise a mixture of viscous solution and physical gel; however, either the properties of the viscous solution or the properties of the physical gel will dominate the composition at a given temperature, pH, salt concentration and/or frequency at which a mechanical stress is applied. G′ is defined as the in-phase shear modulus the value of which equals the vector projection of an applied stress onto the strain vector divided by the strain. G′ represents energy stored elastically which can be completely recovered. G″ is defined as the out-of-phase shear modulus the value of which equals the vector projection of an applied stress on the axis perpendicular to the strain vector divided by the strain. G″ represents energy dissipated (often as heat) which cannot be recovered by the system (see Aklonis and MacKnight (1983) “Introduction to polymer viscoelasticity” 2^{nd} edition. John Wiley and Sons: New York. pg 15-19).

As used herein the phrase “PB polypeptides do not serve to covalently cross-link the polymer” means that the PB polypeptides do not directly link monomers of the polymer to one another via covalent bonding at a rate greater than 100% divided by the number of functional groups on the polymer. Thus, for example, if a 4-arm polyethylene glycol (PEG) polymer is used (4 functional groups), then the maximum cross-link density is 25%, if an 8-arm PEG is used (8 functional groups), then the maximum cross-link density is 12.5%, and so forth. This cross-link density does not provide a cross-linked polymer network, but instead provides elongated polymers. In the case that some PB polypeptides covalently bind to more than one functional site of the polymer but do not serve to covalently cross-link the polymer, these PB polypeptides can still associate with negatively charged polysaccharides within the composition. The actual crosslinking of these PB polypeptides to the polymer is not integral to the structure of the composition; however, these PB polypeptides still function to coordinate physical gelation through non-covalent interactions with the negatively charged polysaccharides.

Thus, the PB polypeptides serve as a bridge between the polymer and the negatively charged polysaccharide; the ionic coordination and/or other noncovalent interactions between the PB polypeptide and the negatively charged polysaccharide serves to maintain the physical structure of the composition.

The negatively charged polysaccharides can consist of multiple molecules of a single type of polysaccharide, or may comprise more than one type of polysaccharide, in which case it is not required (although it is preferred) that each type of polysaccharide has a negative charge. The negatively charged polysaccharides can comprise or consist of polysaccharides that are inherently negatively charged (including, but not limited to, heparin, heparan sulfate, dermatan sulfate, dextran sulfate, keratan sulfate, chondroitin sulfate, alginate, fucan and hyaluronic acid), or can comprise or consist of polysaccharides that are derivatized to be negatively charged (including, but not limited to dextran, dermatan, and agarose that have been chemically modified to contain at least one negatively charged chemical moiety including but not limited to sulfate, phosphate and carboxylic groups).

In non-limiting examples, the negatively charged polysaccharides may comprise sulfated polysaccharides, phosphorylated polysaccharides, and carboxylated polysaccharides. The greater the negative charge of the negatively charged polysaccharide, the more ionic coordination among the components in the resulting composition. Thus, sulfated polysaccharides are an especially preferred embodiment of the negatively charged polysaccharides.

In further preferred embodiments, the one or more negatively charged polysaccharides are selected from the group consisting of heparin, heparan sulfate, dextran sulfate, dermatan sulfate, chondroitin sulfate, keratan sulfate, fucan, alginate, hyaluronic acid, and derivatives thereof. In a most preferred embodiment, the negatively charged polysaccharide comprises one or more polysaccharides selected from the group consisting of heparin, heparan sulfate, dextran sulfate, dermatan sulfate, keratan sulfate and chondroitin sulfate, and derivatives thereof.

The choice of negatively charged polysaccharide will also depend on the type of composition desired. For example, the use of chondroitin sulfate as the predominant or only negatively charged polysaccharide in the composition tends to result in the formation of a viscous solution. In contrast, the use of heparin or dextran sulfate as the predominant or only negatively charged polysaccharide in the composition tends to result in the formation of a physical gel. Some negatively charged polysaccharides, such as chondroitin sulfate, might not contain sufficient charge density to allow for the formation of a physical gel regardless of the concentration of the polysaccharide and the relative ratio between polymer-PB polypeptide and polysaccharide. These mixtures tend to form viscous solutions, especially when using higher molecular weight polysaccharides (such as those with a molecular weight greater than 30,000 Da). Other negatively charged polysaccharides, such as heparin or dextran sulfate, do contain sufficient charge density to allow for the formation of a physical gel. However, the formation of physical gels does not depend solely on the concentration of the polysaccharide or even the relative ratio between polymer-PB polypeptide and polysaccharide. Environmental conditions, such as pH, temperature, salt concentrations, etc., can affect the form of the composition. In addition, the molecular weight of the negatively charged polysaccharide can influence the behavior of the composition. Low molecular weight polysaccharides (such as those with molecular weights below 10,000 Da) might not contain enough binding sites to coordinate physical gelation. In combination with polymer-PB polypeptide molecules with higher molecular weights (such as greater than 30,000 g/mol), mixtures containing low molecular weight polysaccharides, such as heparin (avg. MW 6,000 Da), could form viscous solutions.

Further details of the negatively charged polysaccharides are discussed below.

As used herein, the term “polysaccharide binding polypeptides” (“PB polypeptides”) means polypeptides that bind to negatively charged polysaccharides. Thus, in one embodiment, the polypeptides are any cationic polypeptide, which is defined as a polypeptide with a net charge of at least +1 under at least certain environmental conditions. While it is preferred that the PB polypeptides are cationic under all conditions, the composition may also comprise PB polypeptides that only become cationic within certain pH ranges or salt concentrations. The PB polypeptide may also comprise polypeptides that are modified to possess cationic groups. The PB polypeptides may comprise or consist of a plurality of a single such PB polypeptide, or they may comprise a plurality of each of two or more different polypeptides species.

In a preferred embodiment, the PB polypeptides comprise at least one basic amino acid residue, and more preferably comprise more than one basic amino acid residue, wherein such basic amino acid residues include, but are not limited to, lysine and arginine.

While the PB polypeptides of the invention can be of any length that is capable of use in forming the compositions of the invention, smaller polypeptides are generally preferred from a production and quality control standpoint. Thus, in a preferred embodiment, the PB polypeptides are between 3 and 70 amino acids in length; in a more preferred embodiment, they are between 8 and 40 amino acids in length, and in a most preferred embodiment, they are between 10 and 25 amino acids in length.

The PB polypeptides can be naturally occurring sequences, derivatives thereof, or synthetic sequences with affinities to polysaccharide. Examples of such PB polypeptides comprise or consist of, but are not limited to, polypeptides with amino acid sequences selected from the group consisting of the amino acid sequences shown in Table 1:

TABLE 1

PB Polypeptides and their derivatives from native proteins

| | |
|---|---|
| Amino acid Sequence | Source |

| | | |
|---|---|---|
| KAFAKLAARLYRKA | (SEQ ID NO:1) | Antithrombin III (AT III); |
| (Tyler-Cross, 1994) | | human (PBD1) |
| AAFAKLAARLYRKA | (SEQ ID NO:2) | Modified from human AT III |
| (Tyler-Cross, 1994) | | (PBD2) |
| KAFAALAARLYRKA | (SEQ ID NO:3) | Modified from human AT III |
| (Tyler-Cross, 1994) | | (PBD3) |
| YKKIIKKL | (SEQ ID NO:4) | Platelet Factor TV; human |
| KHKGRDVILKKDVR | (SEQ ID NO:5) | NCAM; human |
| YEKPGSPPREVVPRPRPV | (SEQ ID NO:6) | Fibronectin; human |
| KNNNQKSEPLIGRKKT | (SEQ ID NO:7) | Fibronectin; human |
| RYVVLPRPVCFEKGMNYTVR | (SEQ ID NO:8) | Laminin; porcine |
| (Charonis, 1988) | | |
| KPGPRGQR | (SEQ ID NO:9) | Collagen V; human |
| (Delacoux, 1998) | | |
| KDPKR; | (SEQ ID NO:10) | bFGF; human |
| RSRK (Krewson, 1996) | (SEQ ID NO:11) | |
| YGRKKRRQRRR | (SEQ ID NO:12) | TAT; viral (HIV) |
| (Ho, 2001) | | (PTD1) |
| YARKARRQAIRR | (SEQ ID NO:13) | PTD-3; derivative of TAT |
| (Ho, 2001) | | |
| YARAAARQARA | (SEQ ID NO:14) | PTD-4; derivative of TAT |
| (Ho, 2001) | | |
| YARAARRAARR | (SEQ ID NO:15) | PTD-5; derivative of TAT |
| (Ho, 2001) | | |
| YARAARRAARA | (SEQ ID NO:16) | PTD-6; derivative of TAT |
| (Ho, 2001) | | |
| YAPRRRRRRRR | (SEQ ID NO:17) | PTD-7; derivative of TAT |
| (Ho, 2001) | | |
| YAAARRRRRRR | (SEQ ID NO:18) | PTD-8; derivative of TAT |
| (Ho, 2001) | | |

Other PB polypeptides comprise or consist of amino acid sequences including, but not limited to,

| | |
|---|---|
| PBD4 | |
| KAFAKLAARLYRAA; | (SEQ ID NO:19) |
| PBD5 | |
| KAFAKLAAQLYRKA; | (SEQ ID NO:20) |
| PBD7 | |
| WKA(β)FAKLNCRLYRKA; | (SEQ ID NO:21) |
| PBD8 | |
| AKLNCRLYRKANTKISSKL; | (SEQ ID NO:22) |
| PBD9 | |
| FHRRIKALHRRVKIFHRRIKA; | (SEQ ID NO:23) |
| PBD11 | |
| WKA(β)FAKLAARLYRKA; | (SEQ ID NO:24) |
| PBD12 | |
| FHRRIKAFHRRIKAFHRRIKA; | (SEQ ID NO:25) |
| PBD13 | |
| FHRRIKA; | (SEQ ID NO:26) |
| GRKKRRQRRRPPQ; | (SEQ ID NO:27) |
| AYARAAARQARA; | (SEQ ID NO:28) |
| DAATATRGRSAASRPTERPRAPARSASRPRRPVE; | (SEQ ID NO:29) |
| GWTLNSAGYLLGLINLKALAALAKKIL; | (SEQ ID NO:30) |
| KLALKLALKALKAALKLA; | (SEQ ID NO:31) |
| KIETWWETWWTEWSQPKIKIKRKV; | (SEQ ID NO:32) |
| KAFAKLAARLYRKAGC; | (SEQ ID NO:33) |
| KAFAKLAARLYRAAGC; | (SEQ ID NO:34) |
| AAFAKLAARLYRKAGC; | (SEQ ID NO:35) |
| KAFAALAARLYRKAGC; | (SEQ ID NO:36) |
| KAFAKLAAQLYRKAGC; and | (SEQ ID NO:37) |
| AGGGGYGRKKRRQRRR; | (SEQ ID NO:38) |
| FHRRIKA; | (SEQ ID NO:39) |
| LNRRAKL; | (SEQ ID NO:40) |
| LHRRVKI; | (SEQ ID NO:41) |
| FHRRIKL; | (SEQ ID NO:42) |
| DAATATRGRSAASRPTERPRAPARSASRPRRPVG; | (SEQ ID NO:43) |
| RQIKIWFQNRRMKWKK; | (SEQ ID NO:44) |
| poly-Lys | (SEQ ID NO:45) |
| and poly-Arg | (SEQ ID NO:46) |

sequences (preferably 4-9 residues each);

| | |
|---|---|
| FHRRIKAFHRRIKA; | (SEQ ID NO:47) |
| FHRRJKAFHRRIKAFHRRIKAFHRRIKA; | (SEQ ID NO:48) |
| KAFAKLAARLYRKAGC; | (SEQ ID NO:49) |
| AAFAKLAARLYRKAGC; | (SEQ ID NO:50) |
| A(β)GGGGYGRKKRRQRRR; and | (SEQ ID NO:51) |
| KAFAKILAARLYRKAGC. | (SEQ ID NO:58) |

Such PB polypeptides may further comprise modifications of the above sequences. In non-limiting examples, any “A(β)” residues (where A(β) is beta alanine) can be an “A” residue; any “A” residue can be an “A(β)” residue; and the C-terminus of can comprise carboxylic acid or amide moieties. As a further part of the present invention, it has been discovered that the addition of hydrophobic residues to the PB polypeptides leads to their increased solubility and to increased heparin affinity. Thus, the PB polypeptides may also be modified by adding one or more hydrophobic residues to the therapeutic agent. Non-limiting examples of such hydrophobic groups include FITC-A(β), dansyl-G, dansyl X (where X is any amino acid), any fluorescent label, acetyl group, tryptophan, phenylalanine, and Fmoc or other N-terminal protecting group (e.g. Boc). Thus, in representative examples, the PB polypeptides may comprise or consist of a sequence selected from the group consisting of

| | |
|---|---|
| dansyl-GKAFAKLAARLYRKAGC; | (SEQ ID NO:52) |
| FITC-A(β)AAFAKLAARLYRKAGC; | (SEQ ID NO:53) |
| FITC-A(β)KAFAALAARLYRKAGC; | (SEQ ID NO:54) |
| FITC-A(β)GGGGYGRKKRRQRRR; | (SEQ ID NO:59) |
| Dansyl-GYGRKKRRQRRR; | (SEQ ID NO:55) |
| FITC-A(β)YGRKKRRQRRR; and | (SEQ ID NO:56) |
| FITC-A(β)KAFAKLAARLYRKAGC. | (SEQ ID NO:57) |

The PB polypeptides can comprise or consist of multiple copies of an individual PB polypeptide sequence, or combinations of different PB polypeptide sequences. For example, 3 copies of SEQ ID NO:25 could be covalently linked; alternatively, 1 copy of each of SEQ ID NOS: 3, 12, and 25 can be covalently linked. Thus, the PB polypeptides can comprise or consist of “N” copies of individual PB polypeptides, wherein “N” is preferably between 1 and 10, and more preferably between 1 and 5.

Furthermore, the PB polypeptides may contain one or more additional amino acid residues with functional groups that allow for other functionalities, including but not limited to covalent attachment to the polymer cell adhesion, protease degradation, or extracellular matrix molecule binding.

Thus, in further preferred embodiments, the PB polypeptides comprise or consist of one or more polypeptides selected from the group consisting of SEQ ID NOS:1 to 59. As discussed above, the PB polypeptides may comprise or consist of a plurality of a single such PB polypeptide, or they may comprise a plurality of each of two or more different polypeptides species.

Further details of the PB polypeptides are discussed below.

As used herein, a polymer is a macromolecular substance composed of one or more repeating atomic groups, called monomers, and includes linear, branched, and cross-linked polymers, and combinations thereof. The polymer can comprise copolymers, block co-polymers, graft copolymers, alternating copolymers, and random copolymers. As used herein, a co-polymer is a polymer composed of 2 or more different monomer units. Proteins, polysaccharides, DNA, and RNA are all biological copolymers. Poly(lactic acid-co-glycolic acid) is an example of a synthetic co-polymer.

As used herein, a block copolymer is a polymer composed of linear segments containing 1 or more monomers of the same type, which are covalently attached to at least one other segment containing one or more monomers of a different type. Examples of such block co-polymers include, but are not limited to, co-polymers of ethylene glycol and propylene glycol.

As used herein, a graft copolymer comprises one or more polymer chain to which are covalently attached, along their backbone, one or more linear or branched chains containing one or more monomer unit.

As used herein, an alternating copolymer comprises polymer chains containing either alternating monomers of a different type or alternating blocks of monomers of different type.

As used herein, a random copolymer comprises two or more monomer units that do not occur along the backbone in an alternating fashion. Poly(acrylamide-co-N-isopropyl acrylamide) is an example of a random copolymer.

As used herein, a branched polymer has a non-linear arrangement of monomers. Examples of such branched polymers include polyethylene glycol (PEG) star polymers, PEG comb polymers, and dendrimers.

In a preferred embodiment, the composition comprises a hydrophilic polymer. In a further preferred embodiment, the composition also comprises cationic PB polypeptides and negatively charged polysaccharides, both of which are hydrophilic. A hydrophilic polymer allows the PB polypeptides to associate more freely with the polysaccharides, and provides an increased degree of hydration of the composition, which leads to a more homogenous composition (i.e. less water is excluded). Examples of such hydrophilic polymers include, but are not limited to, polymers comprising or consisting of poly(ethylene glycol) (PEG), poly(ethylene oxide), poly(vinyl alcohol), poly(acrylic acid), poly(ethylene-co-vinyl alcohol), poly(vinyl pyrrolidone), poly(ethyloxazoline), poly(ethylene oxide)-co-poly(propylene oxide) block copolymers, polymethacrylate, polyfumerates, poly(n-isopropylacrylamide), dextran, hyaluronic acid and elastomeric polypeptides or derivatives thereof.

In a further preferred embodiment, the polymer is not charged. As used herein, a “charged” polymer refers to an inherently charged polymer, and does not include a polymer that only becomes charged under specific environmental conditions (such as pH) including but not limited to polymers with oxygen, hydroxyl, and carboxyl residues (such as poly(vinyl alcohol) polymethacrylate, polyfumerates, poly(n-isopropylacrylamide), and poly(vinyl pyrrolidone).

To confer or add additional degradability to the polymer, the polymers can be modified with one or more blocks comprising one or more degradable moieties polymerized on one or both ends of the polymer. For example, poly(ethylene glycol) is not inherently degradable; however, the ends of the poly(ethylene glycol) chains can be modified with degradable polyesters. The PB polypeptides can be covalently bound to the degradable polyesters. Once the degradable polyester degrades, the PB polypeptides are released from the polymer, and the composition formed through interaction of the PB polypeptides and the negatively charged polysaccharides falls apart.

Alternatively, the polymer can be polymerized with degradable oligomers to form degradable block co-polymers, which can serve to increase the loss of coordination of the composition, leading to its degradation. For example, the block copolymers described above can be made with non-degradable polymers and a degradable block, e.g. poly(ethylene glycol)-degradable block-poly(propylene glycol). These blocks can be composed of, for example, lactic acid, glycolic acid, ε-caprolactone, lactic-co-glycolic acid oligomers, trimethylene carbonate, anhydrides, and amino acids. This list is not exhaustive; other oligomers may also be used for block copolymers. The blocks do not have to be hydrophilic, so long as the overall polymer remains hydrophilic.

The polymer may comprise or consist of protein subunits. The protein can be a naturally occurring, chemically synthesized, or recombinant protein. Proteins may be especially useful where additional degradability of the composition is desired, since the protein portion of the polymer will be subject to proteolysis. In addition, such proteins can be selected or engineered to include other desirable features for a given application, including but not limited to binding sites for cells or other proteins. Especially preferred proteins for use in the polymer include collagens, laminins, fibronectin, albumin, and vitronectin. In a further preferred embodiment, a protein polymer does not comprise fibrin.

Where the polymer comprises or consists of protein subunits, the PB polypeptides can be covalently bound to the protein, via, for example, the functional groups on cysteine, tyrosine, and/or lysine residues. Alternatively, when the polymer comprises or consists of protein subunits, the protein subunit can be selected or engineered to include one or more PB polypeptides within the protein sequence. This embodiment may permit tighter control of the relative ratio of polymer to PB polypeptide to negatively charged polysaccharide compared to the embodiments described above.

When the polymer comprises PEG, a preferred embodiment is that the PEG (linear, block or branched) has an average molecular weight of 100 g/mol to 10,000,000 g/mol; more preferred with an average molecular weight of 2000 g/mol to 100,000 g/mol; and most preferred with an average molecular weight of 3000 g/mol to 80,000 g/mol. Similar molecular weight guidelines are appropriate for most synthetic polymers. For dextran, the most preferred embodiment is between 10,000 g/mol to 80,000 g/mol.

As the molecular weight of the polymer increases, the viscosity of the composition will naturally increase. An increase in the viscosity, however, does not imply that the composition can only be in the form of a viscous solution. If a polymer has relatively few sites to which the PB polypeptides can covalently bind, then compositions with larger molecular weight polymers would have a decreased “crosslink” density. As a result, such compositions are more likely to be in the form of a viscous solution. For example, a 4-arm PEG (avg. MW 10,000 g/mol) covalently attached to four PB polypeptides can form a physical gel when mixed with heparin or dextran sulfate. A similar composition comprising 4-arm PEG with an average molecular weight of 100,000 g/mol covalently attached to four PB polypeptides, would have a lower cross link density. This composition might only take the form of a viscous solution. Polymers such as dextran contain many potential sites to which a polypeptide can be coupled. The number of binding sites can scale with size for any polymer composed of monomers with free functional groups. For example, dextran can be modified such that there are three sites per monomer to which a polypeptide could be covalently bound. As a result, a dextran molecule with an average molecular weight of 70,000 g/mol can have several hundred polypeptides covalently bound to it. As a result, the crosslink density is very high, which is likely to result in the formation of a physical gel when mixed with a negatively charged polysaccharide.

Further details of the polymer are discussed below.

In a preferred embodiment, the composition further comprises one or more therapeutic agents that interact with one or more of the negatively charged polysaccharides and the polymer, and are thus sequestered within the composition as a result of the coordination of the composition components. In a more preferred embodiment, the one or more therapeutic agents interact with the negatively charged polysaccharides. As a result of diffusion and loss of coordination of the composition (which can be controlled, as discussed below), the therapeutic agent is released in a desired manner. Dissociation of the therapeutic from the composition occurs due to one or more of the following: therapeutic release from the negatively charged polysaccharide, release of the negatively charged polysaccharide from the PB polypeptides, PB polypeptides from the polymer, combined release of the therapeutic and the negatively charged polysaccharide from the polymer, and combined release of the therapeutic, polysaccharide, and PB polypeptides (as a complex) from the polymer.

As used herein, a “therapeutic agent” means one or more compound of any type that can be used to provide a clinical benefit to a patient. Such therapeutic agents will either inherently interact with negatively charged polysaccharides (ie: they will be cationic) or will be modified to interact with negatively charged polysaccharides. While it is preferred that the therapeutic agents are cationic under all conditions, the composition may also comprise therapeutic agents that only become cationic within certain pH ranges or salt concentrations, or that contain domains/regions that are or can become cationic within certain pH ranges or salt concentrations. Such therapeutic agents include, but are not limited to, nucleic acids, polypeptides, antibodies, and any other chemical or biological compounds. The one or more therapeutic agents may comprise or consist of a plurality of a single such therapeutic agent, they may comprise a plurality of each of two or more different therapeutic agents.

Where the therapeutic agent is modified to be cationic, it is preferred that the one or more therapeutic agents are linked to a transduction domain. The therapeutic agent also could be modified to be cationic by covalently attaching a cationic polypeptide such as polyarginine or polylysine. Furthermore, chemical modifications of the therapeutic agent, such as amination, could make the therapeutic agent cationic. As used herein, the term “transduction domain” means one or more amino acid sequence or any other molecule that can carry a biological or chemical agent across cell membranes. See, for example, Mai et al., Journal of Biological Chemistry 277:30208-30218 (2002). Transduction domains have been demonstrated to be useful to transport, for example, full length proteins, polypeptides, oligonucleotides, bacteriophages, liposomes, and iron nanoparticles. Polypeptides from the HIV protein TAT have been extensively characterized for their transducing ability. (Rusanti, 1997).

Transduction domains can be linked to other therapeutic agents to direct movement of the linked therapeutic agent across cell membranes. In a preferred embodiment, the transduction domain is linked to a polypeptide therapeutic agent via peptide bonding. (See, for example, Cell 55: 1179-1188, 1988; Cell 55: 1189-1193, 1988; Proc Natl Acad Sci USA 91: 664-668, 1994; Science 285: 1569-1572, 1999; J Biol Chem 276: 3254-3261, 2001; and Cancer Res 61: 474-477, 2001).

As a part of the present invention, the inventors have discovered that polypeptides that bind to negatively charged polysaccharides can function as transduction domains. Thus, examples of such transduction domains include, but are not limited to, polypeptides comprising the amino acid sequences shown in SEQ ID NOS:1-59.

Thus, the one or more therapeutic agents may comprise the PB polypeptides described above. As a result, the composition may comprise polypeptides that are used both as PB polypeptides to coordinate the composition, and also as the therapeutic agent, which interacts with the negatively charged polysaccharide but is not bound to the polymer. Alternatively, one or more of the one or more therapeutic agents can be covalently linked to the polymer.

The greater the affinity of the PB polypeptide for heparin (and thus, in general, for the negatively charged polysaccharide of the composition), the more stable the coordination of the composition due to the ionic interaction between the PB polypeptide and the negatively charged polysaccharide. Thus, for applications where it is desired to provide a slower release of one or more therapeutic agents sequestered in the negatively charged polysaccharide, it is preferred to use PB polypeptides with higher binding affinities to heparin. It should be noted that the one or more therapeutic agent will still be released at a slow rate from such compositions based on simple diffusion, but the overall release profile will be more sustained. Alternatively, if a more rapid release rate of the therapeutic agent is desired, PB polypeptides with lower affinity to heparin can be used. However, the PB polypeptides do not have to have high or low affinity for these different release profiles. For example, if both the PB polypeptides and the therapeutic agent have high affinity for heparin, then the therapeutic agent will be released more slowly from the composition. If the PB polypeptides have high affinity for heparin and the therapeutic agent has low affinity for heparin, then the therapeutic agent will be released more rapidly from the composition. If the therapeutic agent is attached to the polymer, the polymer will, in general, dissociate/release from the composition more slowly if it is bound to high affinity PB polypeptides and more rapidly if it is bound through low affinity peptides. Polymer release will also depend on the number of PB polypeptides to which it is attached (the greater the number of PB polypeptides, the more slowly the polymer will release.

Similarly, the one or more therapeutic agents may either inherently possess, or be modified to possess different heparin binding affinities. Thus, for therapeutic agents for which a rapid release from the composition is desired, the therapeutic agent can have, or be modified to have, a relatively low affinity for heparin. In contrast, where a more sustained is desired, the therapeutic can have, or be modified to possess, a higher affinity to heparin. However, the one or more therapeutic agents do not have to have high or low affinity for these different release profiles, as discussed above for the PB polypeptides.

It will be apparent to one of skill in the art that the release profile of the therapeutic agent can also be modified by combining PB polypeptides and therapeutic agents that have different binding affinities for heparin. For example, if a therapeutic agent is used that has a significantly higher affinity for heparin than does the PB polypeptide, then the ionic interaction between the PB polypeptide and the negatively charged polysaccharide in the composition is likely to be interrupted prior to disruption of the interaction between the negatively charged polysaccharide and the therapeutic. In this scenario, the composition will fall apart, and the therapeutic remaining therein will be released as a complex with the negatively charged polysaccharide, which, in at least some cases, have been shown to facilitate transduction of transduction domain-containing polypeptides across cell membranes (Mai et al., JBC 277:30208-30218, 2002). Further such means for modifying the release profile based on the heparin binding affinity of the PB polypeptide and/or the one or more therapeutic agents will be apparent to one of skill in the art.

In a further embodiment of the compositions of the invention, the plurality of PB polypeptides comprise PB polypeptides with high affinities to heparin, and/or the one or more therapeutic agents comprise one or more therapeutic agents with high affinities to heparin. As used herein, the phrase “high affinities to heparin” mean that the PB polypeptide is eluted from a heparin affinity column at NaCl concentrations of 150 mM or more at 25° C. In a preferred embodiment, the PB polypeptide is eluted from a heparin affinity column at NaCl concentrations of between 150 mM and 2M; in a more preferred embodiment between 200 mM and 1 M; in a most preferred embodiment between 250 mM and 850 mM.

For purposes of the present invention, conditions for determining the affinity of the PB polypeptides (and/or the therapeutic agent) for heparin are as follows: The polypeptide is solubilized in 50 mM sodium phosphate buffer, pH 7.5 to a final concentration of 0.5 mg/ml. 1 mg of the polypeptide is loaded onto an Amersham Biosciences HiPrep 16/10 Heparin FF column (or suitable equivalent) at a flow rate of 5 ml/min at 25° C. After loading the polypeptide, a salt gradient is introduced to the column by increasing the sodium chloride content of the buffer from 0 to 2 M NaCl over 20 column volumes. The recorded NaCl concentration required for elution is determined by correlating the maximum absorbance value at 280 and/or 215 nm for each spectrum to the value of the gradient at that time point.

Decreasing the affinity of the therapeutic or the PB polypeptide for the negatively charged polysaccharide can also be accomplished, for example, by altering the polypeptide sequence or by utilizing different negatively charged polysaccharides. For example, polypeptides do not bind as strongly to chondroitin sulfate as to heparin, so they would release more rapidly from compositions in which chondroitin sulfate is the predominate or only negatively charged polysaccharide used, as compared to a composition in which heparin is the predominate or only negatively charged polysaccharide used. Furthermore, decreasing the negatively charged polysaccharides on either a molar basis or on a molecular weight basis would decrease the number of possible binding sites for the therapeutic, and thus would decrease the amount of therapeutic bound, leading to a more rapid release, assuming that the polymer and PB polypeptide content remained the same.

In a further embodiment, a mixture of therapeutic agents with different affinities to the negatively charged polysaccharides can be used, wherein the different therapeutic agents release from the composition at different rates. For example (referring to Table 5), mixtures of PBD1-A (SEQ ID NO:58) and PBD8 (SEQ ID NO:23) could be added to the composition. Since the affinity of PBD1-A for heparin is lower than that of PBD8, PBD1-A will release from the composition more rapidly than PB8. In a further example, a given composition can deliver “X” amount of a therapeutic agent in a controlled manner, where “X” is defined by the number of binding sites that are available on the negatively charged polysaccharide. If 50% of the negatively charged polysaccharide (ie: 0.5× of the binding sites) is chondroitin sulfate, and the other 50% of the negatively charged polysaccharide (ie: 0.5× of the binding sites) is heparin, then the bound therapeutic agent that binds to chondroitin sulfate (ie: 50% of the bound therapeutic agent, assuming all binding sites are bound by therapeutic agent) will release more rapidly than the remaining 50% of the bound therapeutic agent that is bound to heparin.

As a further part of the present invention, it has been discovered that the addition of hydrophobic residues to the PB polypeptides or therapeutic agents leads to their increased solubility and to increased heparin affinity. Thus, another method for modifying the binding and release of the therapeutic agents from the composition is to add one or more hydrophobic residues to the therapeutic agent. Non-limiting examples of such hydrophobic groups include FITC-A(β), dansyl-G, dansyl X (where X is any amino acid), any fluorescent label, acetyl group, tryptophan, phenylalanine, and Fmoc or other N-terminal protecting group (e.g. Boc).

In embodiments where one or more of the one or more therapeutics are covalently bound to the polymer, the therapeutic agent(s) bound to the polymer is (are) an integral part of the composition. The therapeutic is delivered as the composition dissociates or degrades, as discussed above. It is not necessary that the one or more therapeutic agents is released from the polymer. In this embodiment, it is preferred that the polymer comprises PEG and/or dextran, or variants thereof. In these embodiments, it is also possible to include a further therapeutic agent(s) that interact with the negatively charged polysaccharide, as discussed above, and are not covalently bound to the polymer.

In another aspect, the present invention provides methods for making the compositions of the present invention, comprising:

a) covalently linking a polymer to a PB polypeptide under conditions that inhibit crosslinking of the polymer by the PB polypeptide; and

b) contacting the polymer-PB polypeptide complex with negatively charged polysaccharides under conditions that permit non-covalent interaction between the PB polypeptide and the negatively charged polysaccharides,

wherein such contacting forms the composition.

In a preferred embodiment, the method further comprises contacting the composition with one or more therapeutic agents of interest, whereby the one or more therapeutic agents of interest non-covalently interact with the negatively charged polysaccharides or covalently bind to the polymer, and are thus sequestered in the composition.

In this aspect, the various components of the composition are as described above, and the definition of inhibiting the crosslinking of the polymer by the PB polypeptides is as described above.

The compositions of the invention are made using standard methods in the art. For example, the polymer can be derivatized such that 2 or more sites contain an active functional group to which a PB polypeptide can covalently bind. Functional groups include but are not limited to acrylate, methacrylate, vinyl sulfone, N-hydroxyl succinimide, carbodiimide, maleimide, aldehyde, alkyl halide and anhydride moieties. The functional groups can react with moieties within the PB polypeptide including, but not limited to, amine, thiol, hydroxy, amide and carboxyl moieties. The PB polypeptides may contain one or more additional amino acid residues with functional groups that allow for other functionalities, including but not limited to covalent attachment to the polymer. Non-limiting examples of preferred amino acids to add such other functionalities to the PB polypeptide include cysteine, serine, threonine, tyrosine, lysine, asparagines, arginine, glutamic acid and aspartic acid. Standard chemistries can be used to couple free amine, thiol, hydroxy or carboxylic acid moieties within these amino acids to the polymer. In addition to the functional groups contained within the side chains of amino acids, the PB polypeptides can be covalently bound to the polymer via an N-terminal amine, a C-terminal amide or a C-terminal carboxylic acid. In a preferred embodiment, the polypeptides are covalently bound to the polymer through a free amine on the N-terminus or through the free thiol in a cysteine residue. Standard covalent coupling chemistries often involve amine or thiol groups. Since standard polypeptide synthesis involves the deprotection of all amino acid side groups during cleavage from the solid resin support, covalent coupling through amines might lead to covalent bonding of the functional amine of lysine or arginine residues to the polymer. PB polypeptides contain basic residues in order to interact with negatively charged polysaccharides. Covalent bonding via lysine or arginine residues might lead to some random attachment of the PB polypeptide to the polymer. As a result, it is preferred that the PB polypeptides are covalently bound to the polymer via the free thiol in cysteine.

The polymer is bound to PB polypeptides via standard chemical reactions, such as Michael-type addition. Other chemistries useful for covalently bonding the PB polypeptides to a polymer include, but are not limited to nucleophilic addition of hydroxyl, thiol or amine moieties within the PB polypeptide to the polymer or the addition of hydroxyl, thiol or amine moieties within the polymer to the PB polypeptides. For Michael-type addition, a preferred embodiment involves PB polypeptides that comprise one cysteine residue and employing slightly alkaline pH (approximately 7.5, for example) reaction conditions. The free thiol in the cysteine residue of the PB polypeptide selectively adds to an unsaturated chemical moiety such as an acrylate or vinyl sulfone group on the polymer. Since, in this embodiment, the PB polypeptides do not contain multiple cysteine resides and the slightly alkaline pH of the reaction is not favorable for nucleophilic addition of free amine groups (e.g. amino terminus, lysine, arginine), the PB polypeptide will not covalently crosslink the polymer.

Alternatively, the PB polypeptides can be synthesized such that the protecting groups on amino acids such as lysine and arginine are not yet removed. Following cleavage from the solid resin support, the free amine of the amino terminus of the polypeptides would be the only amine group available for covalent attachment to the polymer. As a result, the N-terminal amine group could be covalently bound to the polymer via standard chemical reactions, such as Michael-type addition. For Michael-type addition, a higher pH (between 8 and 9 for example) allows the free amine to covalently bind to an unsaturated chemical moiety such as an acrylate or vinyl sulfone group on the polymer. Regardless of the chemistry used for covalent bonding, the protecting groups on the amino acid residues of the PB polypeptides could then be removed. It will be understood to one of skill in the art that other techniques for inhibiting cross-linking of the polymer subunits via the PB polypeptides can be used.

Depending of the type of composition desired, many relative concentrations of PB polypeptides to polymer can be used. For example, four PB polypeptides can be covalently bound to a 4-arm PEG molecule (ie: 4-fold molar excess of PB polypeptides); an 8-fold molar excess of PB polypeptide can be used if employing an 8-arm PEG molecule, etc. Higher relative molar ratios of PB polypeptides to polymer can also be used, followed by dialysis (or other removal) of uncoupled PB polypeptides. Lower relative molar ratios can also be used when it is desirable that not all possible polymer sites be bound with PB polypeptides. For example, it may be desirable to bind therapeutic agents or other compounds (which may or may not bind to negatively charged polysaccharides) to such other sites on the polymer. This provides further functionality to the compositions, as well as a means to increase the half-life of such drugs or other compounds.

The polymer-PB polypeptide mixture is then optionally dialyzed, preferably using a membrane with a molecular weight cutoff that is no more than three times less than the molecular weight of the peptide-polymer molecules. For example, ten peptides (˜2000 Da each) covalently attached to each molecule of dextran (40 kD) results in a molecule with a new molecular weight of around 60 kD. Thus, an appropriate membrane cutoff would be 20 kD or less.

Subsequently, the polymer-PB polypeptide conjugate is reacted under suitable conditions with the negatively charged polysaccharide to form the composition of the invention. Mixing a solution of polymer-PB polypeptide conjugate with a solution of negatively charged polysaccharide is sufficient to form the composition. In a preferred embodiment, the pH of the composition is between 6.0 and 8.5. In a more preferred embodiment, the pH of the composition is between 6.5 and 8.0. The solvent used to solubilize the polymer-PB polypeptide and negatively charged polysaccharide can be water, a saline solution, or a buffered solution including but not limited to phosphate buffer, HEPES buffer, carbonate buffer or citrate buffer. In a preferred embodiment a buffered solution is used to solubilize the composition components. In a further preferred embodiment, the salt concentration of the composition is similar to or identical to physiological salt concentration. For example, phosphate-buffered saline can have a sodium ion level between 135 and 150 mM and a potassium ion level between 2 and 4.5 mM (Dulbecco's phosphate-buffered saline, for example, has a sodium ion level of around 137 mM and a potassium ion level of around 4 mM). In a preferred embodiment, the temperature at which this composition forms a physical gel is between 0 and 60° C. In a more preferred embodiment, the temperature at which this composition forms a physical gel is between 15 and 50° C. In a most preferred embodiment, the temperature at which this composition forms a physical gel is between 25 and 45° C. Additionally, in a preferred embodiment, the temperature at which this composition forms a viscous solution is between 0 and 60° C. In a more preferred embodiment, the temperature at which this composition forms a viscous solution is between 15 and 50° C. The total component content (i.e. the amount of polymer-PB polypeptide and polysaccharide) of the compositions of the invention is preferably between 0.1% and 70% (w/v). In a more preferred embodiment, the composition contains between 0.5% and 40% (w/v) total component content. In a most preferred embodiment, the composition contains between 1% and 15% (w/v) total component content.

The greater the number of coordination sites for the negatively charged polysaccharide, the greater will be the storage modulus. In other words, the greater the number of PB polypeptides, which are covalently attached to the polymer, the greater the number of sites that can be used for physical crosslinking of the PB polypeptides to the negatively charged polysaccharide, and the more rigid the composition. More highly “crosslinked” gels are better suited for tissue engineering applications such as cartilage and bone regeneration, vascular applications, particularly if the material is exposed to blood flow. Such flow can disrupt the physical bonds if a viscous solution is used. Viscous solutions are preferred where spreading is required, e.g. topical applications, application to the mucosa, application to exposed tissues and organs, etc. Viscous solutions or low percentage physical gels (i.e. total component content below 5% (w/v)) are preferred in nerve regeneration where the neurites cannot extend through dense materials.

Molar ratios of PB polypeptides to the molecular weight of the polymer serve, at least in part, to define crosslink density. Another factor that defines crosslink density is the number of PB polypeptides bound to each negatively charged polysaccharide. In the compositions of the present invention, the polymer is bound to more than one PB polypeptide, while a given negatively charged polysaccharide binds to at least two PB polypeptides. In order to use the compositions to deliver or sequester therapeutics or other moieties via interaction with the negatively charged polysaccharides, sites on the negatively charged polysaccharides must be available to bind to the therapeutic agent.

Optimal molar ratios of the various components of the composition depend on the molecular weight of the polymer, the molecular weight of the negatively charged polysaccharide, the molecular weight of the PB polypeptide, and the density of PB polypeptide on the polymer. According to Rusnati et al. (1999), “data indicates that 5-6 saccharide residues represent the minimum requirement for a significant Tat interaction under physiological buffer conditions.” Tyler-Cross (1996) states that a pentasaccharide is the unit structure of heparin that binds to antithrombin III. Rusnati et al. use a 35 amino acid sequence of Tat and suggest that 1 Tat molecule binds per 3750-5000 Da of heparin. Tyler-Cross uses a 15 amino acid sequence of antithrombin III that binds to a 1450 Da pentasaccharide unit. Thus, in general, longer PB polypeptides are preferably used in conjunction with a higher molecular weight negatively charged polysaccharides so that these long PB polypeptides do not saturate all of the binding sites on heparin. While not bound by any specific mechanism, it is preferred that an upper limit of therapeutic agent (when the therapeutic agent comprises a polypeptide) loading is calculated by the following formula: 1 molecule peptide is equal to the molecular weight of the polysaccharide (as measured in Daltons) divided by 1450 Da minus 2 (at least 2 sites are needed to coordinate the gel).

Given that the negatively charged polysaccharide binds to at least two PB polypeptides (as well as a therapeutic agent in certain embodiments of the invention), a preferred molecular weight range for the negatively charged polysaccharide is greater than 3,000 Da with no theoretical upper limit. In a more preferred embodiment, the upper limit is 10,000,000 Da.

A preferred embodiment of the negatively charged polysaccharides comprises a 4-arm PEG molecule to which 4 PB polypeptides are covalently attached. As will be apparent to one of skill in the art, PEG molecules with fewer or more arms can be employed as effectively, utilizing a number of PB polypeptides that could be equal to the number of PEG arms but results in at least two PB polypeptides covalently bound on each PEG molecule. For a composition with a total component content of 10% (w/v), not all combinations of heparin (18 kD) resulted in the formation of a homogenous composition, which is defined as a composition wherein water is not excluded from the composition. Such exclusion can lead to a non-homogenous composition, which is a composition comprising component rich and component poor regions. For example, a gel-like substance (ie: a viscous solution that appears and can behave, to some extent, as a physical gel, but wherein G′ is not larger than G″) formed when the PEG-PB polypeptide and heparin we added together in a 1:3, 1:2, 1:1 4:1 molar ratio, however, water was excluded in each case, leading to a non-homogenous mixture. It is preferred that the compositions of the invention are homogenous compositions

When PEG-peptide and heparin were added at a 3:2, 2:1, 5:2 and 3:1 molar ratio, water was not excluded, and the gels appeared homogenous. Also, PEG-peptide and dextran sulfate (again 10% (w/v) total component content meaning PEG-peptide and dextran sulfate) were added at a 1:2, 1:1, 2:1 and 3:1 molar ratio. The 2:1 and 3:1 molar ratios resulted in homogenous physical gels. As will be apparent to one of skill in the art, appropriate molar ratios will change, depending on the molecular weight of the polymer, the molecular weight of the polysaccharide, and the number of PB polysaccharides bound to the polymer. For example, if 100 PB polypeptides were bound to a dextran polymer, then you may need a molar ratio of 1:25 (or greater) of PB polypeptide-polymer:polysaccharide. As will be apparent to one of skill in the art, compositions that are dominated by viscous behavior at one perturbation frequency and temperature may be a gel, dominated by elastic behavior, at another temperature or frequency. For example, some physical gels behave more as viscous solutions upon temperature, pH, or salt increases, since the increased energy can disrupt the physical interactions between the PB polypeptide and the negatively charged polysaccharide.

In another aspect, the present invention provides methods for drug delivery, comprising incorporating a therapeutic agent of interest into one of the compositions of the invention, and administering it to a patient in need thereof. The compositions of the invention provide for the controlled delivery of the therapeutic based on the specific design of the composition, as described above. The compositions of the present invention are useful for the delivery of any therapeutic to a target tissue or organ. For example, viscous solutions are preferred for use of the compositions where spreading is important, including but not limited to in topical applications used to deliver the therapeutic transdermally (such as for wound healing), transmucosally (application to mucosa), and application to exposed tissues or organs. Physical gels are preferred, for example, for use in vascular applications, particularly with exposure to blood flow. In such applications, the physical gel may be applied to stents, catheters, and vascular or prosthetic grafts, by coating the device with the compositions of the invention to which have been added a therapeutic agent of interest. Similarly, the compositions of the invention can be injected adjacent to a target tissue, tumor, or organ for local release. For example, a viscous solution can be directly injected, while a dual-barrel syringe (or its equivalent) can be used to separate the PB-polypeptide combination and the negatively charged polysaccharide-therapeutic combination, which can then be locally injected for in situ gelation and drug delivery to the target. For use in the methods of the invention, the compositions can be designed to control the release rate of the therapeutic, as discussed above. Thus, the compositions provide for slow release, sustained release, or combinations thereof by utilizing the compositions of the invention.

The compositions of the invention can also be used, for example, as matrices for tissue engineering, such as bone and cartilage engineering. In this embodiment, a physical gel is preferred, which resembles the extracellular matrix, and thus can be used for the growth of artificial bone or cartilage. Various growth factors or other useful agents can be used as the therapeutic agent in this embodiment.

In another embodiment, the present invention provides methods for delivering therapeutics with a high affinity for heparin from a drug delivery device comprising a polymer, a PB polypeptide covalently bound to the polymer, and a negatively charged polysaccharide bound to the PB polypeptide. “High affinity” is defined as above. With respect to this embodiment, the various preferred embodiments of each of the components of the drug delivery device as described above are also applicable. Such devices include, but are not limited, to those disclosed in US published patent application 2002/0146414.

### EXAMPLES

### Example 1

TAT has recently been described to be a heparin binding peptide (Tyagi 2001). The dissociation constant for TAT from heparin is related to the molecular weight of heparin and to the number of TAT molecules bound to an individual heparin molecule. For low molecular weight heparin and heparin to which only one molecule of TAT is bound, the K_{D} is approximately 0.7 μM (Rusnati, 1999). This value is one order of magnitude lower than that of wild type antithrombin III, and suggests that the heparin binding properties of TAT can be used to develop polysaccharide-based delivery systems.

The peptides listed in Table 2 were synthesized in the ASU Protein Chemistry Laboratory using standard solid state F-moc chemistry. Following synthesis, the β-alanine in the TAT sequence was labeled with FITC using an isothiocyanate chemistry. Peptide purification was performed using an acetonitrile/water gradient with a C4 reverse phase column on an ÄKTA FPLC.

TABLE 2

Synthesized peptides with affinity to heparin

| | | Relative |
|---|---|---|
| Heparin-Binding | | Affinity |
| Domain | Amino Acid Seguence* | to Heparin |
| HBD-high | KAFAKLAARLYRKAGC | 100% |
| | (SEQ ID NO:49) | |
| HBD-low | AAFAKLAARLYRKAGC | 14.3% |
| | (SEQ ID NO:50) | |
| TAT | A(β)GGGGYGRKKRRQRRR | 170% |
| | (SEQ ID NO:51) | |

*The relative affinity measurements correspond to the bold-faced amino acids.

A 10-fold molar excess of HBD-high and HBD-low each were covalently bound, via Michael-type addition, to acrylated dextran (40 kD; 31% degree of substitution) to make 10% solutions (w/v). The conjugation proceeded in Tris-buffered saline (TBS), pH 7.4 at 37° C. for 4 hours. Following conjugation, each solution was centrifuge filtered using a 3,000 Da molecular weight cutoff membrane and reconstituted with TBS, pH 7.4. As seen in Table 3, conjugated and unconjugated dextran solutions were made with and without heparin (6,000 MW). These stock solutions were then transferred to a 96-well plate in 100 μl aliquots. A 2,2-dimethoxy-2-phenylacetophenone initiator was added to each well, and the solutions were allowed to gel for 20 minutes in the presence of ultraviolet light.

TABLE 3
Pre-gel solutions

| | | | Number |
|---|---|---|---|
| Type of Gel | Amount of Dextran | Amount of Heparin* | of Gels |
| HBD high- | 0.5% dextran-HBD high | 20 molar equivalent | 14 |
| heparin | | | |
| HBD high | 0.5% dextran-HBD high | None | 6 |
| HBD low- | 0.5% dextran-HBD low | 20 molar equivalent | 14 |
| heparin | | | |
| HBD low | 0.5% dextran-HBD low | None | 6 |
| Dextran- | 0.5% dextran | 20 molar equivalent | 4 |
| heparin | | | |
| Dextran | 0.5% dextran | None | 4 |

*Heparin was added at a 20 molar excess relative to the amount of unconjugated or conjugated dextran.

Following polymerization, each gel was washed either with 100 μl of a FITC-labeled TAT solution (7.25 mg/ml) or 100 μl TBS, pH 7.4. Then, the gels were incubated at 37° C. for 1 hour. After 1 hour, 100 μl of equilibrated solution was removed from each gel and replaced with 100 μl TBS, pH 7.4. The gels were incubated at 37° C. 100 μl samples were taken again at 3.5, 5, 6.5, 8.5, 16 and 28 hours following the initial TAT/TBS washing.

A BioRad Fluoromark instrument was used to measure 538 nm emission using a 485 nm excitation. Measurements for each experimental sample was compared with the linear range of a FITC-labeled TAT standard curve to determine TAT concentration.

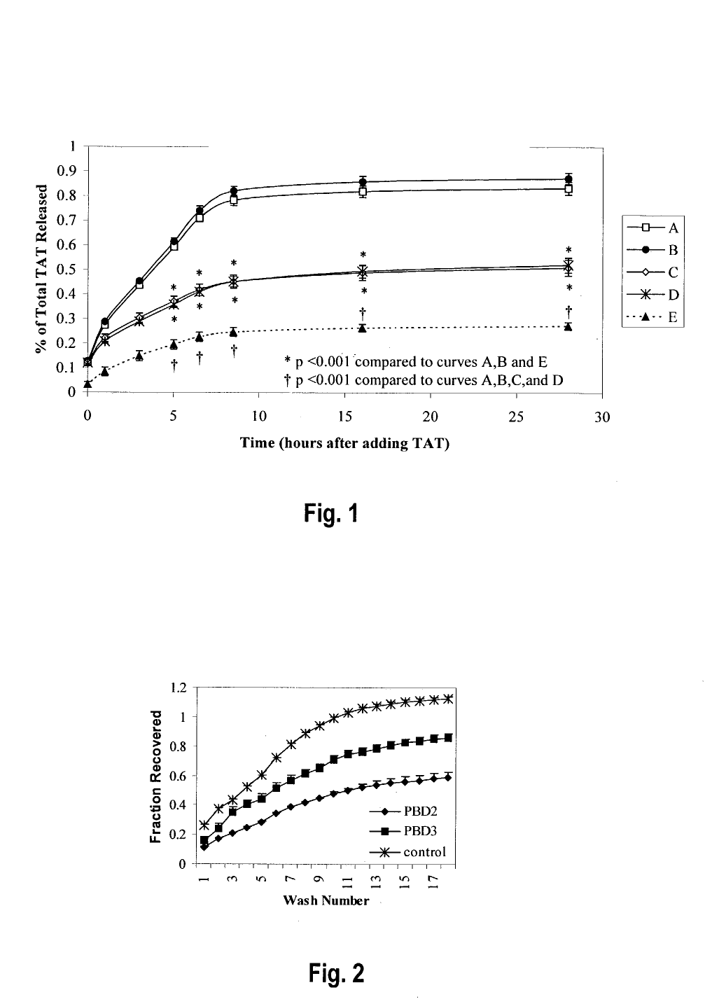

FIG. 1 shows the results of the experiment. The results of the FITC-labeled TAT release experiment are shown over a course of 28 hours following the addition of the TAT to the gels. The letters in the legend correspond to sample groups in the following manner: A—HBD high-TAT; B—HBD low-TAT; C—HBD high-heparin-TAT; D—HBD low-heparin-TAT; E—dextran-heparin-TAT. Curves D and B, HBD low- and HBD high-coupled dextran with added TAT, show the fastest release with all of the available TAT being released within 7 hours. Curves A and C, HBD low- and HBD high-coupled dextran with added heparin and TAT, show slower release rates of TAT. As seen in curve E, dextran with added heparin and TAT shows little to no release. The heparin and TAT can form insoluble complexes that do not allow for release of TAT if 4 or more TAT molecules bind per heparin molecule. This phenomenon is especially observed in case E since no heparin binding domains exist within the dextran gel. In addition, due to its size, the heparin remains trapped within the gel. As a result, heparin within gels without heparin-binding domains conjugated to the dextran are free to associate with more TAT molecules than heparin within gels containing other heparin binding sites. Some of the TAT in cases A and C also appears to be tightly bound to the heparin in a precipitated state.

In this example, a photoinitiated dextran was used to encapsulate heparin and give a proof-of-concept that the delivery/sequestration of heparin-binding peptides (e.g. TAT) could be controlled. The heparin most likely was entrapped in the gel and could not diffuse out well. The gels may have been overloaded with peptide relative to the binding ability of the gels (e.g. not enough heparin) As a result, the peptide recovered from the first 7 hours could have been peptide that never bound within the gel. The data show that shows that heparin entrapped within a gel can sequester heparin binding peptides relative to gels not containing heparin

### Example 2

A 4-fold molar excess of HBD-high was covalently bound, via Micheal-type addition, to poly(ethylene glycol tetravinyl sulfone) (VS-PEG) to make 10% solutions (w/v). The conjugation proceeded in phosphate buffered saline (PBS), 0.05 M EDTA, pH 8.0 at 37° C. for 4 hours. 7 mg heparin (6,000 MW) was then added to the HBD-high-VS-PEG complex to allow for two heparin molecules per one molecule of the HBD-high conjugated VS-PEG. The solution was allowed to incubate overnight at room temperature. After incubation, the solution appeared opaque and non-flowing. It behaved as a physical gel. In this example, the heparin acted as a bridge between heparin-binding domains on different HBD-high VS-PEG molecules, which resulted in the formation of a physical gel.

### Example 3

In examples 3 and 4, PBD₁ is dansyl-GKAFAKLAARLYRKAGC (SEQ ID NO:52), PBD₂ is FITC-A(β)AAFAKLAARLYRKAGC (SEQ ID NO:53) PBD₃ is FITC-A(β)KAFAALAARLYRKAGC (SEQ ID NO:54) and TAT is FITC-A(β)GGGGYGRKKRRQRRR (SEQ ID NO:59). Also, PEG₁₀-4 refers to 4-arm poly(ethylene glycol) (avg. MW 10,000 g/mol). PEG₁₀-4PBD1 refers to a molecule consisting of 4 PBD1 peptides conjugated to a 4-arm PEG (10,000 g/mol avg. MW). One could similarly use, for example, a 4-arm PEG (20,000 g/mol) with 2 or 3 peptides attached; a 4-arm PEG (10,000 g/mol) 2-4 peptides attached; an 8-arm PEG (either 20,000 or 40,000 g/mol) with 2-8 peptides attached; other molecular weights of PEG; PEG molecules with more than 4 or 8 arms; and linear PEG.

Preliminary diffusion studies were performed by forming 10% 100 μl gels (2:1 heparin: PEG₁₀-4PBD₁) in the bottom of a 96-well plate. Soluble PBD₂ or PBD₃ (1:4 peptide:heparin) was added to the gels and allowed to incubate for one hour at room temperature. Control solutions consisted of PEG₁₀-4PBD₁ and PBD₃ without heparin. After incubation, the gels (n=6 of each type) were washed several times with PBS, pH 7.4 by adding 100 μl PBS and removing 100 μl of the solution. The extracted solution was examined for the presence of FITC label. Standard curves of PBD₂ and PBD₃ were used to calculate the fraction of each type of peptide removed during the wash steps.

FIG. 2 shows the results of the preliminary diffusion studies. After 18 washes, all of the PBD₃ peptide in the control solutions had been recovered. The PBD₂ and PBD₃ peptides both had slower release profiles indicating that the heparin-based gels were effective in sequestering these peptides. After 18 washes, approximately 80% of the PBD₃ peptide had been recovered whereas only about 60% of the PBD₂ peptide was recovered. Different release profiles were expected since PBD₃ has a dissociation constant 1.8 times higher than that of PBD₂. These results indicate that the gels can release heparin-binding peptides based at different rates based upon heparin affinity.

### Example 4

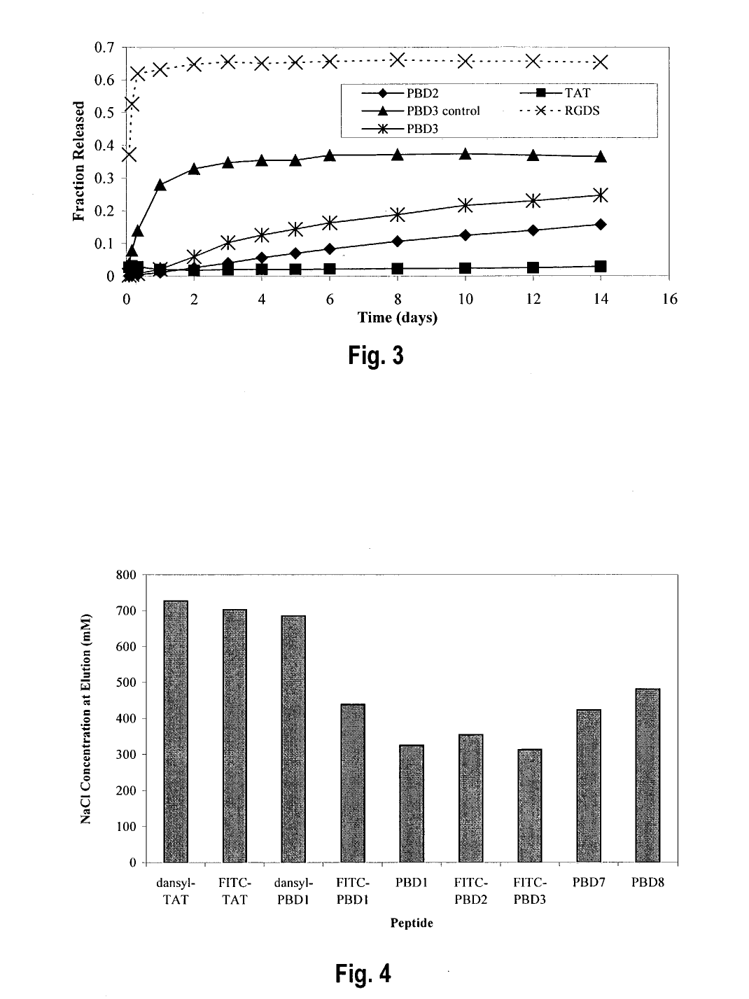

Preliminary diffusion studies were performed by forming 10% 50 μl gels (1:2 heparin: PBD₁-conjugated 4 arm PEG) in dispodialyzers. Each formed gel also contained either TAT, PBD2 or PBD3 to be released (2 peptides per heparin molecule incorporated into the gel) Controls consisted of gels containing RGDS (SEQ ID NO:60), which was not expected to associate with the gel and which would diffuse freely. A second control was made with peptide-conjugated PEG and PBD3 alone so that PBD3 diffusion would not be affected by binding to heparin. The dispodialyzers then were placed into 15 ml tubes containing 15 ml of phosphate buffered saline (PBS). At varying time points over a 2-week period, 500 μl aliquots were taken from the PBS reservoir and replaced with new PBS. Results of this study are show in FIG. 3. As expected, both the RGDS peptide and the PBD3 peptide free from heparin were able to diffuse rapidly out of the gel. After one day, almost all of the RGDS peptide had been released. The free PBD3 control peptide also released at a fast rate since no heparin was present to slow diffusion. All gels containing heparin released heparin-binding peptides at rates slower than those of controls. Almost none of the TAT peptide was recovered after 2 weeks. PBD3 had a faster diffusion rate than PBD2, and this difference correlates well with the relative heparin affinity of each peptide.

The other peptides were released more slowly from the gel and their rate of release was dependent on their affinity for heparin. Table 4 shows initial rates of release for each of the peptides. The release rates also confirm that peptide release can be controlled by affinity to heparin.

TABLE 4
Initial release rates of heparin-binding peptides from heparin-binding gels

| | Initial Release Rate | |
|---|---|---|
| Peptide | (% of total peptide released per day) | Affinity to Heparin (K_{D}) |
| RGDS | 93 | |
| (SEQ | | |
| ID | | |
| NO:60) | | |
| PBD3 | 25 | |
| control | | |
| PBD2 | 1.3 | 3.7 ± 0.6 × 10⁻⁷ M [1] |
| PBD3 | 3.4 | 6.6 ± 0.1 × 10⁻⁷ M [1] |
| TAT | 0.04 | 3.0 ± 1.4 × 10⁻⁸ M [2] |

In this example, some of the peptide was trapped in the upper chamber, so not all of the peptide could be recovered. Consequently, it appears that only the RGDS peptide was fully recovered. This result may explain why it appears other peptides have plateaued at recovery concentrations well below 90-100%. However, between the peptides studies, the relative rates of release over time are valid. For example, the TAT peptide did not release from the gels even after 2 weeks.

### Example 5

FIG. 4 is a chart depicting the relative heparin affinity of various heparin-binding peptides. Each peptide was solubilized in 50 mM sodium phosphate buffer, pH 7.5 to a final concentration of 0.5 mg/ml. Then, 1 mg of each peptide was loaded onto an Amersham Biosciences HiPrep 16/10 Heparin FF column at a flow rate of 5 ml/min. After loading the peptide, a salt gradient was introduced to the column by increasing the sodium chloride content of the buffer from 0 to 2 M NaCl over 20 column volumes. The recorded NaCl concentration required for elution was determined by correlating the maximum absorbance value at 280 and/or 215 nm for each spectrum to the value of the gradient at that time point. Table 5 shows the sequence information for each peptide as well as NaCl concentration at the time of elution.

TABLE 5

Relative heparin affinity of
heparin-binding peptides.

| | | NaCl Elution |
|---|---|---|
| Peptide | Sequence | Conc. (mM) |
| Dansyl-TAT | Dansyl-GYGRKKRRQRRR | 727.6 |
| | (SEQ ID NO:55) | |
| FITC-TAT | FITC-A(β)YGRKKRRQRRR | 703.6 |
| | (SEQ ID NO:56) | |
| Dansyl-PBD1 | Dansyl-GKAFAKLAARLYRKAGC | 685.9 |
| | (SEQ ID NO:52) | |
| FITC-PBD1 | FITC-A(β)KAFAKLAARLYRKAGC | 438.8 |
| | (SEQ ID NO:57) | |
| PBD1-A | KAFAKLAARLYRKAGC | 325.0 |
| | (SEQ ID NO:58) | |
| FITC-PBD2 | FITC-A(β)AAFAKLAARLYRKAGC | 354.8 |
| | (SEQ ID NO:53) | |
| FITC-PBD3 | FITC-A(β)KAFAALAARLYRKAGC | 313.0 |
| | (SEQ ID NO:54) | |
| PBD7 | WKA(β)FAKLNCRLYRKA | 422.6 |
| | (SEQ ID NO:22) | |
| PBD8 | AKLNCRLYRKANKSSKL | 481.2 |
| | (SEQ ID NO:23) | |

FIG. 4 and Table 5 show the salt concentration measured at the time of elution for each of several heparin-binding peptides. Of particular interest is the trend that the dansyl and FITC TAT peptides have very similar elution profiles. In contrast, FITC-PBD1 elutes at a much lower salt concentration than dansyl-PBD1. Unlabeled PBD1 has an even lower salt threshold required for elution. Both dansyl and FITC are considered hydrophobic moieties; however, it has been observed that the addition of these groups to the N-terminus of many heparin-binding peptides improves solubility in aqueous environments. In addition, the above data suggests that the addition of these fluorophores can alter considerably the properties (e.g. heparin affinity) of a given peptide. When PBD1 was synthesized with a tryptophan residue at the N-terminus in place of the FITC-A(β) or dansyl-G motif, the solubility of the peptide was improved as was the heparin affinity; however, the heparin affinity was not as high as the same peptide synthesized with an N-terminal FITC or dansyl.

### Example 6

### Examples of Mechanical Properties

To covalently attach the peptide dansyl-PBD1 (dansyl-GKAFAKLAARLYRKAGC) (SEQ ID NO:52) to the 4-arm PEG vinyl sulfone (avg. MW 10,000 g/mol), two 2 ml 20% solutions were created containing a 4:1 peptide:PEG molar ratio in phosphate-buffered saline (PBS), pH 7.4 and 5 mM EDTA, pH 8.0. Due to residual acidity of the peptide, the pH of the solutions was raised to pH 7.4 using triethanolamine. These solutions then were incubated at 37° C. for 12 hours. Following covalent attachment, the 2 ml solutions were transferred into dialysis membranes (molecular weight cutoff of 3,500 g/mol) and dialyzed against PBS, pH 7.4 for 12 hours. Following dialysis, the concentrations of both solutions were calculated to be 8.3%. These solutions will be referred to as PEG₁₀-4PBD1.

To create the compositions, several combinations of heparin (MW 18,000 Da) and PEG₁₀-4PBD1 were combined in Eppendorf tubes such that the final concentration of the combined component content was 10% in 50 μl. These combinations consisted of the following molar ratios of PEG₁₀-4PBD1 to heparin: 1:2, 2:3, 1:1, 3:2, 2:1, 5:2 and 3:1. For each PEG-peptide:heparin mixture, a gel-like material formed immediately upon the addition of heparin to the PEG₁₀-4PBD1; however, in most cases, the 50 μl solutions contained a component rich and component poor region. After centrifuging each tube for 30 seconds at 18,000×g, only the gels containing PEG₁₀-4PBD1:heparin ratios of 2:1, 5:2 and 3:1 did not show evidence of water exclusion. The gels having the least water exclusion were those containing a ratio of 2:1 PEG₁₀-4PBD1:heparin. This ratio of PEG-peptide:heparin also results in homogenous gels with a higher polysaccharide content than the 5:2 and 3:1 gels. Physical gels (50 μl volume) were also created with dextran sulfate (avg. MW 15,000 g/mol) in a 1:2, 2:3, 1:1, 2:1 and 3:1. Similar results were observed with the dextran sulfate gels since all combinations formed a gel-like material; however, only the 2:1 and 3:1 PEG₁₀-4PBD1:dextran sulfate gels did not exclude much water.

To characterize dynamic mechanical properties of the compositions of the invention, 500 μl 10% physical gels (2:1 PEG₁₀-4PBD1:heparin and 2:1 PEG₁₀-4PBD1:dextran sulfate) were formed on the surface of a rheometer. Then, a 20 mm acrylic parallel plate rheometer geometry was lowered to a gap distance of 1 mm. Any gel pushed out by the descending geometry was removed with a spatula. Dynamic mechanical testing was performed by applying a 1 Pa oscillatory stress during a frequency sweep from 1 to 100 rad/s at 25° C. In total, four separate physical gels were formed and tested.

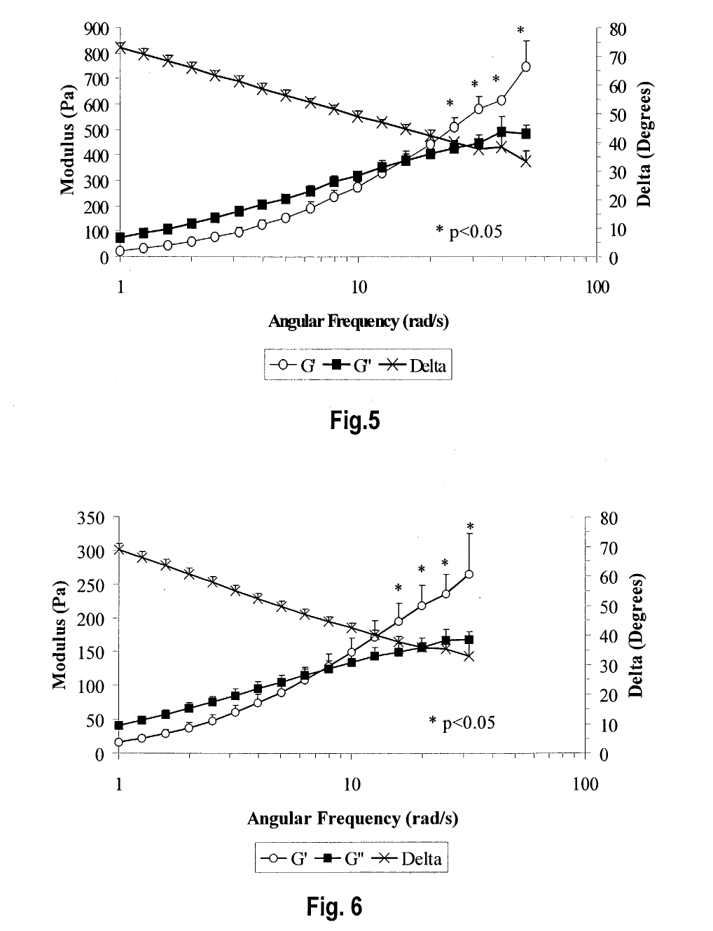

FIGS. 5 and 6, heparin and dextran sulfate compositions, respectively, show that the measured mechanical moduli were repeatable between different gels (as suggested by the standard deviation). FIG. 5 shows the plotted storage modulus, G′, and the loss modulus, G″, from the average of four separate compositions. The gelation point (as indicated when G′ and G″ are equivalent, which corresponds to the measurement delta having a value of 45 degrees) occurred at 15 rad/s. The heparin/PEG₁₀-4PBD1 mixtures had gel-like properties between 15 and 50 rad/s. Below 15 rad/s, the loss modulus, G″, was higher than the storage modulus, G′, indicating the dominance of viscous behavior. Since the gels are physically associated and not chemically coupled, slow angular frequency perturbations would allow the physically associated PEG₁₀-4PBD1 and heparin molecules to assume characteristics typical of viscous solutions. From 15-50 rad/s, G′ became larger than G″ showing the formation of a physical gel as indicated by the presence of more elastic-like characteristics. Above 50 rad/s, the angular frequencies were too high, and the gels dissociated (data not shown). Overall, these dynamic mechanical properties seemed reasonable, especially since they were only an order of magnitude lower than those reported for a 40% covalently-linked gel (Elbert, D. L., et al., “Protein delivery from materials formed by self-selective conjugate addition reactions”. Journal of Controlled Release, 2001.76(1-2): 11-25).

FIG. 6 shows the frequency-dependent behavior of dextran sulfate-based compositions as the average of four separate compositions. The gelation point (as indicated when G′ and G″ are equivalent, which corresponds to the measurement delta having a value of 45 degrees) occurred at 8 rad/s. The dextran sulfate/PEG10-4PBD1 mixtures had gel-like properties between 8 and 32 rad/s. Below 8 rad/s, the behavior of the mixture was dominated by viscous behavior. Above 32 rad/s, the gels dissociated (data not shown). For both heparin and dextran sulfate gels, high angular frequencies caused gel dissociation. However, due to the physical nature of gelation, the gels quickly reformed when the oscillatory stress at high frequencies was removed.

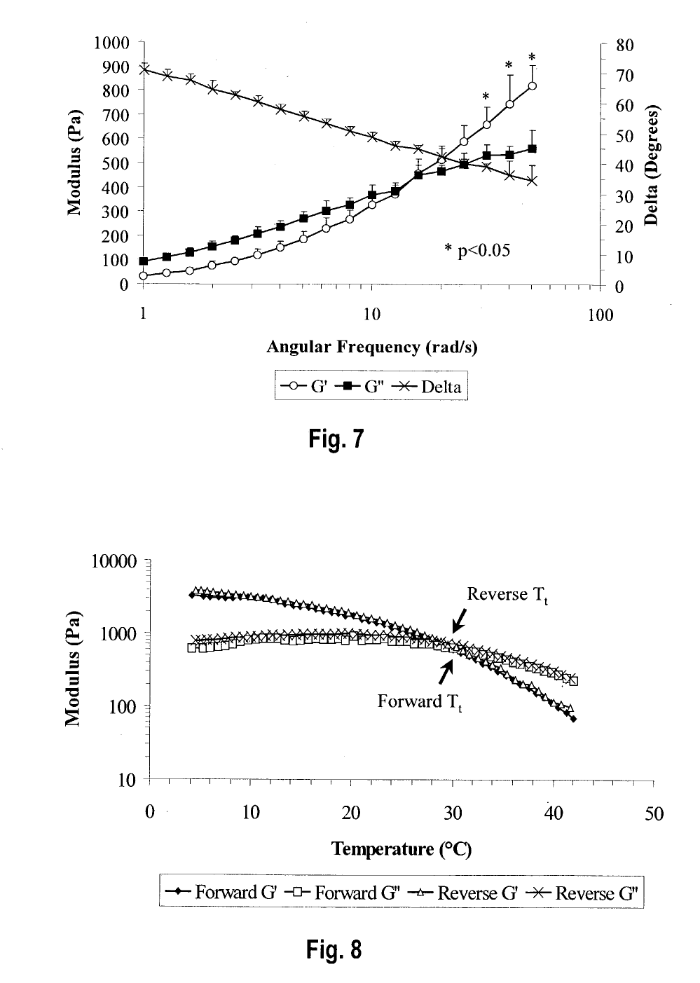

FIG. 7 gives an example of the physical reversibility of heparin-based compositions. The plotted storage modulus, G′, and the loss modulus, G″, are the average of four consecutive runs of one 10% gel consisting of 2:1 PEG₁₀-4PBD1:heparin. Each run consisted of a frequency sweep (using a 1 Pa oscillatory stress) from 1 to 100 rad/s. After the completion of each run, the gel was allowed to rest for 30 seconds before the next frequency sweep began. In all cases, the gels dissociated at frequencies above 50 rad/s. This plot shows that the polysaccharide-based physical gels could reform quickly and give reproducible mechanical properties following the removal of high, disruptive angular frequencies. From 32 rad/s to 50 rad/s, the storage modulus, G′, was significantly higher than the loss modulus, G″ (p<0.05).

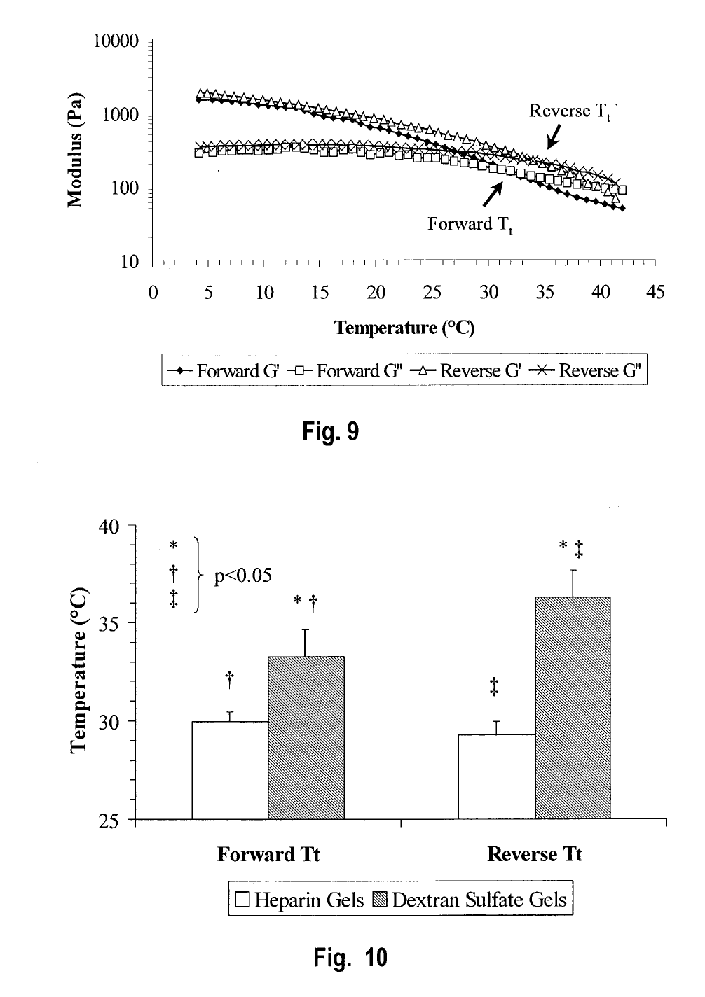

To evaluate the effect of temperature on the mechanical properties of the heparin-based and dextran sulfate-based compositions, a temperature sweep was performed on each of the four 10% compositions used in the frequency sweep examples mentioned previously (in all cases, the temperature sweep was performed after the frequency sweep data had been collected). Temperature sweeps were performed by applying a 1 Pa oscillatory stress at an angular frequency of 20 rad/s. The values for the temperature during the sweep ranged from 4° C. to 42° C. For each gel, the temperature of the rheometer plate was lowered to 4° C. After equilibrating for 2 minutes, the storage and loss moduli were recorded as the temperature increased at a rate of 5° C. per minute. Once the temperature reached 42° C., the rheometer plate began to cool at the same rate. Thus, the forward and reverse temperature profile of the physical gel was recorded. FIG. 8 and FIG. 9 show a plot of the storage and loss modulus as a function of temperature for one of the measured heparin (FIG. 8) and dextran sulfate compositions (FIG. 9). In both experiments, the storage and loss moduli were recorded for one gel as the temperature rose from 4° C. to 42° C. and then decreased back to 4° C. The transition temperature (the point at which G′ and G″ were equivalent), Tₜ, occurred around 29° C. for both the forward and reverse sweeps for the heparin composition, and around 33° C. during the forward sweep and around 36° C. during the reverse sweep for the dextran sulfate composition. Above the Tₜ, the mixture retained some mechanical integrity, but acted more as a viscous solution.

As the data shows, these compositions are thermally reversible. Since the mechanism of gelation is physical, the ionic and hydrogen bonding within the composition should dissociate at elevated temperatures and reassociate as the temperature decreases. In each plot, the transition temperature, Tₜ, was defined as the temperature at which the value of the storage modulus, G′, equals that of the loss modulus, G″. Although the compositions were no longer considered true gels above Tₜ, the materials did not lose all mechanical strength. Rather, the compositions behaved as viscous solutions. After performing temperature sweeps for 4 heparin-based and 4 dextran sulfate-based compositions, the average transition temperature for forward and reverse temperature sweeps were compared. As seen in FIG. 10, the heparin-based compositions had a forward and reverse Tₜ around 29° C. Differences between forward and reverse Tₜ were not significant. The forward Tₜ for dextran sulfate-based gels was around 33° C. This Tₜ was significantly higher than the forward Tₜ for heparin-based gels. In addition, the forward Tₜ for dextran sulfate-based gels was significantly different than the 36° C. reverse Tₜ for dextran sulfate-based gels.

We claim:

1. A composition comprising:
a) a polymer network;
b) a plurality of polysaccharide binding (PB) polypeptides, wherein the plurality of PB polypeptides are covalently bound to the polymer network, but wherein the PB polypeptides do not serve to covalently cross-link the polymer network; and
c) negatively charged polysaccharides non-covalently bound to the plurality of PB polypeptides.

2. The composition of claim 1 wherein the composition comprises a physical gel.

3. The composition of claim 1 wherein the composition comprises a viscous solution.

4. The composition of claim 1 wherein the negatively charged polysaccharides comprise one or more polysaccharides selected from the group consisting of sulfated polysaccharides, phosphorylated polysaccharides, and carboxylated polysaccharides.

5. The composition of claim 1 wherein the negatively charged polysaccharides comprise one or more polysaccharides selected from the group consisting of heparin, heparan sulfate, dextran sulfate, dermatan sulfate, chondroitin sulfate, keratan sulfate, fucan, alginate, and hyaluronic acid.

6. The composition of claim 1 wherein the negatively charged polysaccharides comprise one or more polysaccharides selected from the group consisting of heparin, heparan sulfate, dextran sulfate, dermatan sulfate, keratan sulfate and chondroitin sulfate.

7. The composition of claim 1 wherein the plurality of PB polypeptides comprise cationic polypeptides.

8. The composition of claim 1 wherein the plurality of PB polypeptides comprise polypeptides of between 3 and 70 amino acids in length.

9. The composition of claim 1 wherein the PB polypeptides comprise one or more amino acid sequence selected from the group consisting of SEQ ID NOS: 1-59.

10. The composition of claim 1 wherein the composition comprises a single PB polypeptide species.

11. The composition of claim 1 wherein the composition comprises two or more PB polypeptide species.

12. The composition of claim 1 wherein the polymer is hydrophilic.

13. The composition of claim 12 wherein the PB polypeptide and the negatively charged polysaccharide are hydrophilic.

14. The composition of claim 1 wherein the polymer is not charged.

15. The composition of claim 1 wherein the polymer comprises one or more of poly(ethylene glycol) (PEG), poly(ethylene oxide), poly(vinyl alcohol), poly(acrylic acid), poly(ethylene-co-vinyl alcohol), poly(vinyl pyrrolidone), poly(ethyloxazoline), poly(ethylene oxide)-co-poly(propylene oxide) block copolymers, polymethacrylate, polyfumerates, poly(n-isopropylacrylamide), dextran, hyaluronic acid and elastomeric polypeptides.

16. The composition of claim 1, wherein the composition further comprises one or more therapeutic agents that interacts with one or more of the negatively charged polysaccharide and the polymer.

17. The composition of claim 16 wherein at least one therapeutic agent non-covalently interacts with the negatively charged polysaccharide.

18. The composition of claim 16 wherein at least one therapeutic agent covalently binds to the polymer.

19. The composition of claim 16 wherein the one or more therapeutic agents comprise at least two therapeutic agents.

20. The composition of claim 19 wherein the at least two therapeutic agents possess different heparin binding affinities.

21. The composition of claim 16, wherein the one or more therapeutic agents comprise a transduction domain.

22. The composition of claim 1 wherein the plurality of PB polypeptides comprise PB polypeptides that elute from a heparin affinity column at an NaCl concentrations of 150 mM or more.

23. The composition of claim 16 wherein the one or more therapeutic agents comprise PB polypeptides that elute from a heparin affinity column at a NaCl concentrations of 150 mM or more.

24. A method for drug delivery, comprising administering one or more therapeutic agents to a patient in need thereof, wherein the therapeutic agent is delivered by using the composition of claim 1.

25. A method for making a composition, comprising:
a) covalently linking a polymer to a PB polypeptide under conditions that inhibit crosslinking of the polymer by the PB polypeptide; and
b) contacting the polymer-PB polypeptide complex with negatively charged polysaccharides under conditions that permit non-covalent interaction between the PB polypeptide and the negatively charged polysaccharides.

26. The method of claim 25 further comprising contacting the composition with one or more therapeutic agents of interest, whereby the one or more therapeutic agents of interest non-covalently interact with the negatively charged polysaccharides, or covalently bind to the polymer, and are sequestered in the composition.

27. A method for drug delivery, comprising delivering therapeutics that elute from a heparin affinity column at an NaCl concentrations of 150 mM or more from a drug delivery device that comprises a polymer, a PB polypeptide covalently bound to the polymer, and a negatively charged polysaccharide bound to the PB polypeptide.
